(19) United States
(12) Patent Application Publication
(10) Pub. No.: US 2016/0354240 A1
(43) Pub. Date: Dec. 8, 2016

(54) Automated Eye Drop Instillation

(71) Applicants: Carrie Chauhan, (US); University of Florida Research Foundation, Inc., Gainesville, FL (US)

(72) Inventors: Anuj Chauhan, Gainesville, FL (US); Carrie Chauhan, Gainesville, FL (US)

(21) Appl. No.: 15/238,111

(22) Filed: Aug. 16, 2016

Related U.S. Application Data
(63) Continuation-in-part of application No. PCT/US2015/016109, filed on Feb. 17, 2015.
(60) Provisional application No. 61/940,645, filed on Feb. 17, 2014.

(51) Int. Cl. A61F 9/00

(52) U.S. Cl. CPC A61F 9/0026; A61F 2009/0043

(57) ABSTRACT

A device for the delivery of eye drops to one or both eyes of a patient includes a frame for placement over the patient's eyes, a holder for a commercially available eye drop bottle and a fluid delivery means. The eye drop delivery device can include a video camera to monitor the opening and closing of the eyes and a processor or a connector to a remote processor with software to receive input from the camera and deliver output to the delivery means to start and stop delivery of eye drops to the patient's eye.

### CROSS-REFERENCE TO RELATED APPLICATIONS

This is a continuation-in-part of International patent application No. PCT/US2015/016109, filed Feb. 15, 2015, which claims the benefit of U.S. Provisional Application Ser. No. 61/940,645, filed Feb. 17, 2014, the disclosure of which is hereby incorporated by reference in its entirety, including all figures, tables and amino acid or nucleic acid sequences.

### BACKGROUND OF INVENTION

The delivery of medications to the eye can be advantageous in many ways; however, the administration is plagued with challenges. The application of fluids as eye drops has been problematic for many, and is the norm with children who tend to blink or jerk during delivery. Elderly patients often lack the dexterity to correctly position the eye dropper or squeeze bottle for consistent delivery to the eye. The medication is worthless if it lands on the eyelid, nose, forehead, or cheek. Consistent and assured dosage can be extremely difficult to achieve. This method of administrating medicines is inaccurate and wasteful as presently carried out.

Eye droppers and, particularly, eye dropper bottles often poke the patient in the eye, which, in the worst case, can damage the eye and permits the delivery tip to become contaminated with bacteria, viruses, or fungi, which can proliferate in the ophthalmological solution. This can promote subsequent infection in the patient or a second patient that happens to use the solution. Common eye drop technology does not satisfactorily control the amount of medication that is dispensed or ensure delivery.

Accordingly, there remains the need for an ophthalmic delivery device where a consistent volume of fluid is delivered into the eye. A device is needed that avoids under-dosing because the fluid misses the target eye or over-dosing because the patient attempted to compensate for the partial administration by delivering an addition unknowable portion of a dose.

Known devices for delivering ophthalmic formulations to the eye most rely on dispensing the formulations as a mist, aerosol, or very small drops from small nozzles. These devices complicate the typical process of eye drop instillation and frequently require cassettes or reservoirs on non conventional designs for delivery of eye drop formulations. There remains a need to improve the process of eye drop instillation by automating the process, yet with devices that are configured to use common commercially available eye drop bottles.

### BRIEF SUMMARY

Embodiments of the invention are directed to an eye drop delivery device that has a frame configured for placement over a patient's eyes with one or more ports for fixing at least one eye drop bottle to the eye drop delivery device. The frame can include a pair of temples, a bridge, nose pads and a holder for positioning a commercially available eye drop bottle. The eye drop delivery device has a means of detecting orientation, a means of forcing an orientation, or both, to permit release of at least one eye drop only when the means assures a vertical path to the patient's eye. The means of detecting orientation can include at least one accelerometer. The means of forcing an orientation can be a bed mount, which is not necessarily a bed, but is any recliner that comfortably allows the head to be forced into an orientation that the patient's eye is vertically underneath the eye drop delivery device's means of delivering the eye drop to the patient's eye. The means of delivering the eye drop can be a mechanical device that squeezes the eye drop bottle with a reduction of the volume of the eye drop bottle within a period of two seconds or less such that a drop, which may be a spray, is ejected to the patient's eye. The means of delivering the eye drop can employ a plunger, a clamp, or a coil connected to a linear actuator.

The eye drop delivery device can include a processor within or attached to the frame or that is remote to the frame and, optionally, the mechanical body of eye drop delivery device, being connected in a wired or wireless manner, where the processor is configured to receive input signals and deliver output signals as directed by software. The eye drop delivery device can include a means for observing one or both of the patient's eyes to determine if the eye is in open or closed state. The means for observing can be one or more video cameras, lens, or lens and image sensor electronically or electromagnetically connected to provide input signals to the processor. The video camera, lens, or lens and video image sensor can is mounted on the frame and connected to the processor. The eye drop delivery device can include a means of displaying a series of audio cues and/or a series of visual cues in a rhythmic fashion to allow the patient to synchronize blinking such that the processor can generate an output signal for delivering the eye drops from the means of delivering where the input signal from the video camera allows computation that the patient's eye is in the state of being open or to activate the means of delivering at a time predetermined to coincide with a specific number of the audio cues and/or visual cues such that the patient has open eyes for delivery. The eye drop delivery device can include a user interface that can have a means to trigger the means of delivering. The patient initiates a triggered input signal to the processor which subsequently provides the output signal to the means of delivering to release the at least one eye drop on demand. The user interfaced means to trigger can be a hand actuator or a voice actuator. The processor or the user interface can comprise a Wi-Fi enabled cell phone or computer. The processor or the user interface can employ software to output signals to one or more dosage alarming devices to alert the patient to prepare to use the eye drop delivery device. Such an alarming device can also provide the information to a medical practitioner and/or to a pharmacist or other eye drop bottle provider to facilitate treatment of the patient.

### BRIEF DESCRIPTION OF THE DRAWINGS

FIG. 1 shows a drawing of an eye fluid delivery device where drop control and camera control is of a single unit mounted to the frame, according to an embodiment of the invention.

FIG. 2 shows a drawing of an eye fluid delivery device where drop control is separate from camera control and resides as three units that are mounted to the frame, according to an embodiment of the invention.

### DETAILED DISCLOSURE

Embodiments of the invention are directed to a device and method for the delivery of an ophthalmic solution to the eye in a controlled and consistent manner. Additionally, in this manner the contamination of the ophthalmic solution, or subsequently the eye, can be prevented. The device employs a frame, similar to that for eye glasses, where commercial eye drop bottles can be attached into ports in the frame such that only the outer surface of a bottle contacts the device. The eye drop bottle is firmly positioned in the frame to ensure that it does not move during the dispensing process. The patient wearing the device must orient his or her head as when lying down on a bed such that the eye drop bottle is aligned to dispense drops in the direction of gravity. This orientation assures that the drops will flow vertically with a controlled trajectory. Unlike when the drops are delivered while a patient is standing, the vertical acceleration of the drop easily promotes missing the target eye. When the orientation is not vertical, the velocity of the ejected drop must be sufficiently high that the vertical displacement does not cause the eye to be missed. By keeping the eye drop bottle vertical, the drops can be discharged slowly to the target eye. In an embodiment of the invention, detection of a vertical orientation is achieved by integration of three accelerometers in the frame or the dispensing device. By placing three accelerometers in the device the vertical direction can be detected because two of the accelerometers will detect zero and the third will detect the gravitational acceleration (g). Such an approach allows cell phones, computers, and other devices to determine their orientation, but are not used in state of the art eye drop dispensing systems.

In another embodiment of the invention, the eye drop dispensing device is mounted on the frame of a bed, for example, the head board of a bed, such that the vertical alignment is assured when the patient is lying in an appropriate position, for example, on his or her back on the bed. The patient can be situated on a head rest for positioning of the head so that the device is properly aligned with the eye. The dispensing can occur when triggered by the patient using a hand held trigger or an audio command when the device includes a receiver and processor.

Another component of the eye drop dispensing device, according to an embodiment of the invention, is a communication system that informs the drop dispensing device when the eye drop bottle is vertical. The device will communicate to the patient that the eye drop bottle is vertical and ready to dispense eye drops. A sequence of audio or visual cues is communicated to the patient. For example, a red light on the frame can indicate that the bottle is not vertical. A green light announces that the bottle is vertical and allows the patient a period of time to prepare to receive the drops, allowing, for example, one to three seconds for the patient to blink or otherwise prepare. After this period, the light will start flashing telling the patient to be ready for eye drop delivery. The use of visual signals can also act as a cue to the patient that it is time for a dose of the eye drops. Alternatively, such a dosage time cue can be transmitted to a patient's cell phone possessing Wi-Fi capabilities to promote a desirable dosing schedule. Furthermore, such remote alarming capabilities also permit the monitoring of the doses consumption, or lack thereof, for example, by alerting and recording events directly to a medical practitioner that prescribed or is to monitor the eye drop treatment. The device can also monitor the quantity of the eye drop fluid in the eye drop bottle, by any means, including tracking the consumption over time, the resistance to compression of the bottle, or any other measurement that indicted consumption of eye drops, such that the patient and/or a pharmacy can be alerted to the need for a new eye drop bottle.

According to an embodiment of the invention, the device includes a dispensing system that is designed to eject one drop each time that delivery has been communicated from the eye drop dispensing device. It will be important to ensure that no satellite drops are ejected and to ensure that the ejected eye drop reaches the desired target in the eye. The frames will be designed to allow some flexibility in adjusting the location of the dispensing device to ensure that the vertical oriented eye drop bottles are positioned at a desired location, preferentially pointing at the pupil center or, alternatively, instilled into the lower cul-de-sac when the patient pulls down the lower eyelid. In embodiments of the invention, dispersion can be done either manually or automatically. One or more cameras can be mounted on the glasses to help with automatic alignment. The fluid is provided in a controlled manner to the eye as a drop or, alternately as a spray, such that a prescribed quantity is reliably delivered. Typically, but not necessarily, the fluid outlet of the device will be positioned within about 2.5 cm from the eye to which the fluid is delivered.

In an embodiment of the invention, fluid can be delivered simultaneously to both eyes of a patient. In embodiments of the invention, the fluid is delivered to only one eye or to both eyes, sequentially. In an embodiment of the invention, two eye drop bottles are attached so that eye drops can be delivered simultaneously to both eyes. In another embodiment, four eye drop bottles are attached containing two different formulations so that two types of drugs can be delivered to each eye.

In an embodiment of the invention, the drop creation can be initiated by deforming the eye drop bottles that are ported into the frame. The drop mechanism allows that there is no direct contact with the formulation. In an embodiment of the invention, the eye drop bottle is deformed by a linear actuator or a cam. The linear actuator pushes against the bottle to create deformation that reduces the gas volume inside the bottle leading to an increase in pressure. In an embodiment of the invention, the linear actuator is connected to a string that is coiled around the bottle ending in a fixed support. As the linear actuator pulls on the string, the coils tighten around the bottle applying the compressive force uniformly. In another embodiment of the invention, the bottle is placed in between a jaw, such that the tightening of the jaw creates the compressive force. The drop dispensing permits rapid drop creation. After the bottle is aligned vertically and the signal is received by the dispensing system, the drop is created in less than 1 second. After creation, the drop takes a fraction of a second to reach the eyes. Since the time in between blinks is only a few seconds, it is important to achieve a rapid drop creation. In an embodiment of the invention, the drops are created rapidly, in two seconds or less, for example, in less than one second. The rapidity of the drop dispensing depends on two factors, the rate of deformation of the eye drop bottle and the extent of deformation. In an embodiment of this invention, the drop dispensing system deforms the bottle to reduce the volume by 10% of the total volume in less than one second. The linear actuator triggers very rapidly, and once triggered the actuator accelerates rapidly. The actuator travels a distance that is 5% of its total length to reduce the bottle volume by about 10%.

In an embodiment of the invention, a sensor is included that allows the determination of motion and/or the state of the eye to which the fluid is to be delivered. The sensor can be a motion sensor, or a light, visible or infrared, which can provide a signal when the eyelid is open and the eye is exposed. In an embodiment of the invention, the sensor can rely on video input that is provided by one or more cameras that receive images through one or more lenses that are positioned for continuous observation of one or both eyes. The video camera can be a CCD device, a CMOS device, or any other type of device. The image need not be of high resolution and 0.3 megapixels or less is adequate, although higher resolution is acceptable as long as the processor used for assessing the blinking and actuation of the valve controlling the fluid is sufficiently rapid to effectively perform the necessary calculation in effectively “real time” where the time lag between receiving the images and providing the outlet is sufficiently short, for example, fractions of a second. The frame rate can be 20 or more frames per second (fps), for example, the common 30 to 60 fps of typical video display rates. The common frame rate can permit the actuation of the control valve in as little as, for example, 0.1 seconds. The received images permit the processor to employ software that determines if the eye is or should be in an open state to permit access of a fluid or if the eye is in a closed state that inhibits access to a fluid. The video feed can be received for a period of time that permits calculation of a periodic or quasi periodic sequence of blinks, such that expected periods of fluid access to and restriction from the eye can be predicted and used during the control of the outlet for fluid delivery during a confident access period. The video feed can be used to determine the most rapid rate at which the patient blinks and detect the moment of opening of the eye after a blink. In this manner, the fluid delivery can commence as soon as the eye is accessible to the fluid and is delivered at a rate such that delivery will be halted before the subsequent blink is anticipated, or determined to commence. During this “access window” the aligned fluid outlet assures delivery into the eye and not the eye lid or eye lashes. The continuous video feed can be employed to detect the closing of the eye in real time where the processed signal can evoke an output signal to the control valve of the controlled outlet to halt delivery. The control system can permit the delivery of portions of the dosage over a plurality of access windows until the entire prescribed dose is delivered and then place the controlled outlet in a closed state. If desired, the video imaging can be used to detect physiological changes to the eye, such as dilation or other responses of the eye to the delivered dose, for assurance that the dose is delivered rather than the device attempted delivery from an empty reservoir or an unprimed outlet.

Control of the device can be carried out with a computer. In an embodiment of the invention, the computer can be linked via a cable to the device or, in another embodiment of the invention, a small single board computer; for example, a BeagleBone by Beagleboard can be included into the device. Positional and rotational information can be controlled with the aid of a Gray code. The processor can be a microprocessor that is included as a portion of the frame or can be connected to the camera(s) mounted on the frame. The processor employs imaging software to determine the end and/or commencing of a blink and the blink rate. The processor can be a personal computer, for example, a portable laptop computer, or other microprocessor driven portable device, for example, a tablet computer or a smartphone. The signal between the camera mounted on the frame and the processor can be wired or wireless. The processor has a user interface for input of dosage variables. Dosage variables that can be input include: fluid identity; solute concentration; solution viscosity; prescribed dosage; or any other needed input.

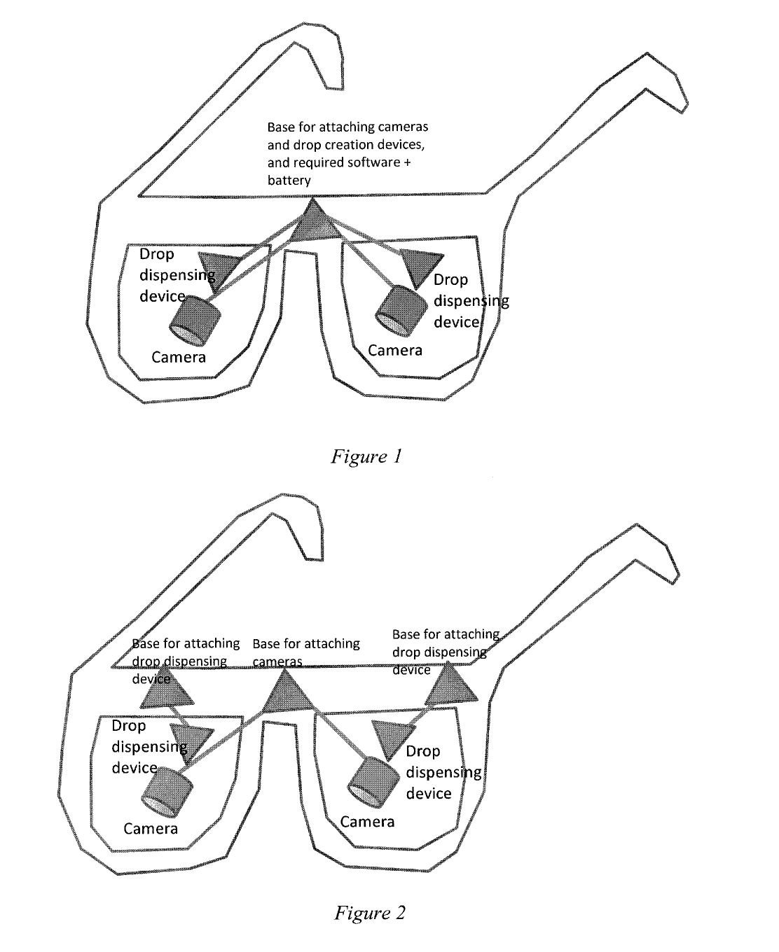

Among the many configurations that the drop delivery and camera features can be combined on the frame of an eye fluid delivery device, two possibilities are illustrated in FIGS. 1 and 2. In FIG. 1, the cameras are mounted to a common portion of the frame with the drop delivery features. The mounting feature can house or support any microprocessor, transmitter, reservoirs, and/or controllers desired. In FIG. 2, the mounting of the camera is separate from a first drop delivery feature and a second drop delivery feature. In this manner, the supporting and controlling portions of the device can be separated on the frame. In an alternate configuration, the camera or the eye observation system may not be attached to the dispersing device, but communicate remotely. For example, a camera phone or a camera attached to a computer could be used for the imaging and communicating the information to the dispensing device mounted on the glasses, through Wi-Fi or radio communication. A positioning stage may be employed in these to facilitate positioning of the head for camera imaging. Many other configurations can be employed, as would be appreciated by one of ordinary skill in the art. In another configuration, both the camera and the dispensing device could be mounted on a stage with a specific location and assembly for placement of the patient's chin.

In another embodiment of the invention, additionally, the video feed can be used to precisely locate the region within the eye where the medication should be delivered and to orient the drop or aerosol producing device to ensure delivery to the desired region of the eye. In another embodiment the video feed can be analyzed to evaluate ocular health, e.g., analyzing redness of the eye.

In another embodiment of the invention, the processor can be replaced by a sequence of audio cues, for example, beeps or similar sounds, where the subject is instructed to close eyes at every cue. After a few cues, the drop creating device can use the cue sequence to synchronize the drop delivery with the timing to assure a fully opened eye. In another embodiment of the invention, the camera and the beeps can be used to enhance the synchronization between drop delivery and eye opening.

It should be understood that the examples and embodiments described herein are for illustrative purposes only and that various modifications or changes in light thereof will be suggested to persons skilled in the art and are to be included within the spirit and purview of this application.

We claim:

1. An eye drop delivery device, comprising:
a frame, wherein said frame is configured for placement over a patient's eyes and at least one port to receive at least one eye drop bottle;
a means of detecting orientation and/or a means of forcing an orientation to permit release of at least one eye drop only when the means assures a vertical path to the patient's eye;
a means of delivering the eye drop to the patient's eye, wherein the means of delivering the eye drop squeezes the eye drop bottle with a reduction of the volume of the eye drop bottle within a period of two seconds or less;
optionally, a processor within or attached to the frame or is remote to the frame;
optionally, a means for observing at least one of the patient's eyes to determine a state of being open and a state of being closed, wherein the means for observing comprises at least one video camera, lens, or lens and image sensor electronically or electromagnetically connected to provide input signals to the processor;
optionally, a means of displaying a series of audio cues and/or a series of visual cues, wherein a rhythm is provided for synchronization of blinking, and wherein the processor has software for providing output signals to actuate the means of delivering the eye drops when the processor has received the input signals from the video camera and has computed that the patient's eye is in the state of being open or at a time predetermined to coincide with a specific number of the audio cues and/or visual cues; and/or
optionally, a user interface including a means to trigger, wherein the patient initiates a triggered input signal to the processor to provide the output signal to the means of delivering to release the at least one eye drop.

2. The eye drop delivery device of claim 1, wherein the frame comprises a pair of temples, a bridge, nose pads and a holder for positioning the eye drop bottle.

3. The eye drop delivery device of claim 1, wherein the means of detecting orientation comprises at least one accelerometer.

4. The eye drop delivery device of claim 1, wherein the means of forcing an orientation comprises a bed mount wherein the patient is positioned in a bed under the eye drop delivery device wherein the patient's eye is at the base of a vertical path from the means of delivering.

5. The eye drop delivery device of claim 1, wherein the video camera is mounted on the frame.

6. The eye drop delivery device of claim 1, wherein the lens or lens and video image sensor is mounted on the frame and connected to the processor.

7. The eye drop delivery device of claim 1, wherein the means of delivering the eye drops comprises a plunger, a clamp, or a coil connected to a linear actuator.

8. The eye drop delivery device of claim 1, wherein the means of delivering delivers the eye drops of about 30 microliter in volume.

9. The eye drop delivery device of claim 1, wherein said means of delivering provides two of the eye drops sequentially in a preselected interval of time.

10. The eye drop delivery device of claim 1, wherein the user interfaced means to trigger is a hand actuator or a voice actuator.

11. The eye drop delivery device of claim 1, wherein the processor or the user interface comprises a Wi-Fi enabled cell phone or computer.

12. The eye drop delivery device of claim 1, wherein the processor or the user interface includes software and output signals for at least one dosage alarming device and/or to a informing and recording device situated by a practitioner and/or eye drop bottle provider.

13. A method of delivering an eye drop to an eye, comprising
providing an eye drop delivery device according to claim 1;
attaching an eye drop bottle to the eye drop delivery device;
inputting dosage information to a processor of the device;
positioning a frame of the eye drop delivery device on a patient in need of the eye drops;
placing the patient in a desired orientation;
monitoring one or both eyes of said patient with at least one video camera of the eye drop delivery device;
optionally, observing the onset and end of a plurality of blinks and calculating the average period of the open eye between consecutive blinks with the processor and/or synchronizing a plurality of blinks with a plurality of audio cues and/or visual cues and detecting a state of being open of the patient's eye after a blink or establishing a blinking rhythm and providing a signal to a means of delivering to initiate delivery of the eye drops to the patient's eye;
delivering the eye drops for a period less than the average period between blinks; and
repeating the steps of observing and calculating and/or synchronizing, and delivering until a prescribed volume of the eye drops to the patient's eye.

14. The method of claim 13, wherein the eye drops comprise a drug, vitamin, or lubricant.
